(19) United States
(12) Patent Application Publication
(10) Pub. No.: US 2014/0118993 A1
(43) Pub. Date: May 1, 2014

(54) ELECTRONIC INCENSE ASSEMBLY AND METHOD FOR MANUFACTURING ELECTRONIC INCENSE ASSEMBLY

(71) Applicant: HON HAI PRECISION INDUSTRY CO., LTD., New Taipei (TW)

(72) Inventor: PO-CHOU CHEN, New Taipei (TW)

(73) Assignee: HON HAI PRECISION INDUSTRY CO., LTD., New Taipei (TW)

(21) Appl. No.: 13/864,279

(22) Filed: Apr. 17, 2013

(30) Foreign Application Priority Data
Oct. 30, 2012 (TW) 101140210

(51) Int. Cl. A61L 9/03; F21V 33/00

(52) U.S. Cl. CPC A61L 9/03; F21V 33/0028
USPC 362/96; 156/245

(57) ABSTRACT

An electronic incense assembly includes an incense stick, a light source and an incense head member. The incense stick includes a top surface, a bottom surface opposite to the top surface, and side surface connecting between the top surface and the bottom surface. The side surface is coated with an opaque layer. The light source faces the bottom surface for emitting light to the bottom surface. The incense head member is formed by molding and is adhered on the top surface.

### BACKGROUND

1. Technical Field

The present disclosure relates to electronic incense assemblies and, particularly, to an electronic incense assembly and a method for manufacturing the electronic incense assembly which can reduce the cost of the electronic incense assembly.

2. Description of Related Art

Electronic incense assemblies usually include an incense stick and a light source. The incense stick is made of transparent material and includes a flat low end and a bulb-shaped upper end appearing as an incense head. The light source faces the low end and a side surface of the incense stick is coated with opaque material. The incense stick guides light emitted from the light source to the upper end to simulate a burning incense. However, the bulb-shaped upper end is usually shaped by manually polishing. It is a low efficiency to manufacture the incense stick and increases the cost of the electronic incense assemblies.

Therefore, it is desirable to provide an electronic incense assembly and a method for manufacturing the electronic assembly, which can overcome the above-mentioned shortcomings.

### BRIEF DESCRIPTION OF THE DRAWINGS

FIG. 1 is an exploded isometric view of an electronic incense assembly, according to a first embodiment.

FIG. 2 is an assembled isometric view of the electronic incense assembly of FIG. 1.

FIG. 3 is a cross-sectional view take along line III-III of the electronic incense assembly of FIG. 2.

FIG. 4 is a cross-sectional view of an electronic incense assembly, according to a second embodiment.

FIGS. 5-9 are schematic views showing successive stages of a method for manufacturing an electronic incense assembly, according to an exemplary embodiment.

### DETAILED DESCRIPTION

Embodiments of the disclosure will be described in detail, with reference to the accompanying drawings.

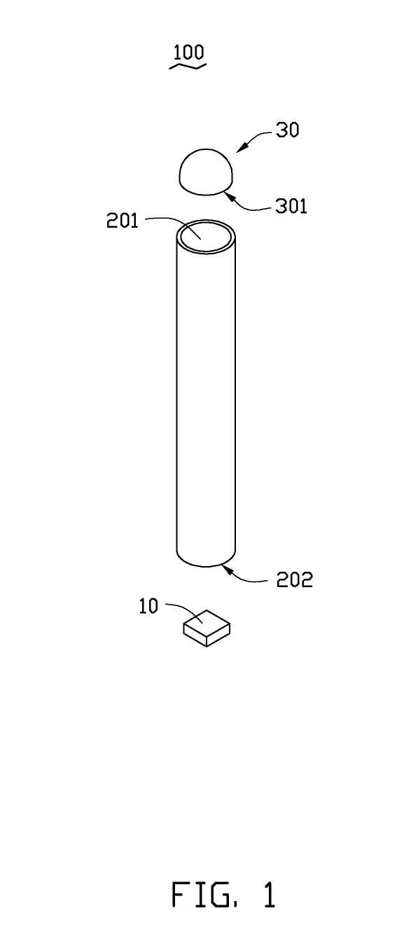

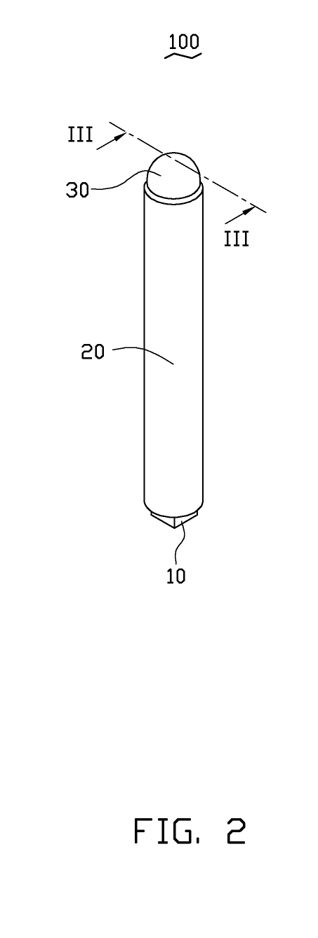

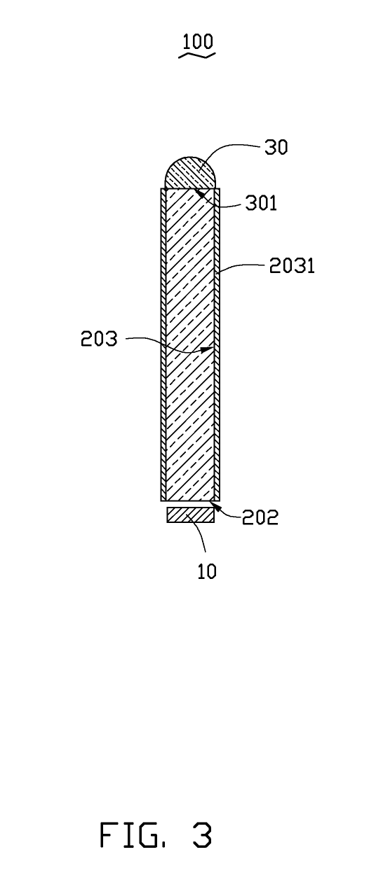

FIGS. 1 to 3 show an electronic incense assembly 100, according to a first embodiment. The electronic incense assembly 100 includes a light source 10, an incense stick 20, and an incense head member 30.

The light source 10 is a light emitting diode (LED). In the embodiment, the light source 10 emits red light.

The incense stick 20 is made of transparent material and is cylindrical. The incense stick 20 can be made of one material selected from poly methyl meth acrylate (PMMA), poly carbonate (PC), silicone, and glass. The incense stick 20 includes a circular top surface 201, a circular bottom surface 202 opposite to the top surface 201, and a side surface 203 perpendicularly connecting between the top surface 201 and the bottom surface 202. An opaque layer 2031 is coated on the side surface 203. The opaque layer 2031 can be black or brown and is made of ink.

The incense head member 30 is bulb-shaped and molded by transparent adhesive. In the embodiment, the incense head member 30 is hemispherical-shaped and molded by ultraviolet curable adhesive. The ultraviolet curable adhesive can be cured when illuminated by ultraviolet light. The incense head member 30 includes a circular contact surface 301. An area of the contact surface 301 corresponds to an area of the top surface 201. The contact surface 301 is totally adhered to the top surface 201 such that the incense stick 20 and the incense head member 30 are connected with each other. In alternative embodiments, the incense head member 30 is semi-oval or cone-shaped. The area of the contact surface 301 is less than the area of the top surface 201.

The light source 10 is positioned to be spaced from and face the bottom surface 202 such that light emitted by the light source 10 is projected to the bottom surface 202.

In use, the light source 10 emits red light, the red light is projected to the bottom surface 202 and introduced into the incense stick 20, then is transmitted to the incense head portion 30 through the incense stick 20, and finally emits from the incense head portion 30 to simulate a burning incense. The opaque layer 2031 can prevent the red light from leaking out from the side surface 203.

The incense head member 30 is molded by adhesive and adhered to the top surface 201. It is high efficiency to manufacture the incense head 30 and decreases the cost of electronic incense assembly 100 relative to shape a bulb-shaped upper end on an incense stick by manually polishing.

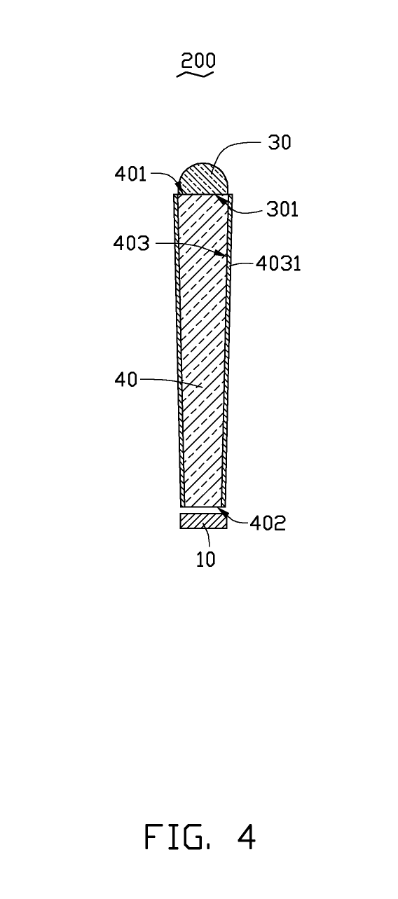

FIG. 4 shows an electronic incense assembly 200, according to a second embodiment. The electronic incense 200 includes a light source 10, an incense stick 40, and an incense head member 30. The incense assembly 200 is similar to the incense 100 except that the incense stick 40 is a truncated-cone. The incense 40 includes a circular top surface 401, a circular bottom surface 402 opposite to the top surface 401, and a side surface 403 connected between the top surface 401 and the bottom surface 402. The top surface 401 is coaxial with the bottom surface 402 and an area of the top surface 401 is greater than an area of the bottom surface 402. An opaque layer 4031 is coated on the side surface 403. The incense head member 30 includes a circular contact surface 301 corresponding to the top surface 401. The contact surface 301 is adhered to the top surface 401 such that the incense stick 40 and the incense head member 30 are connected with each other.

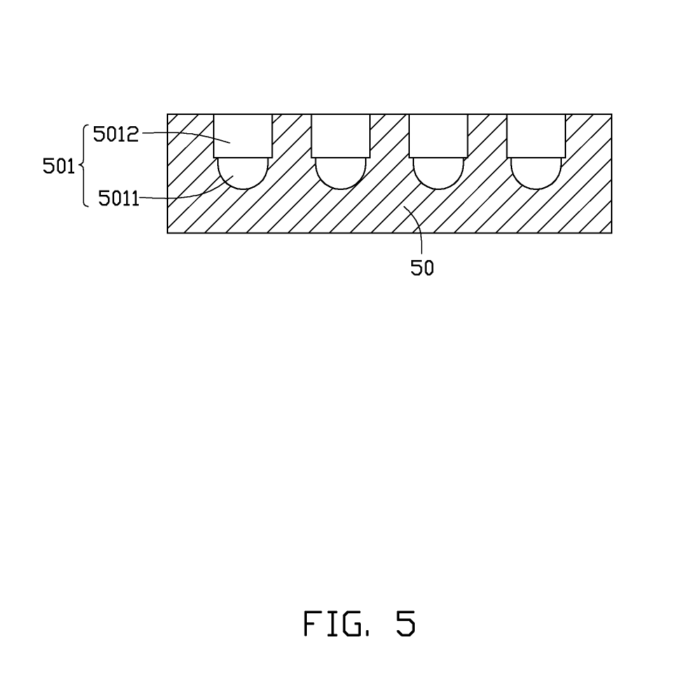

FIGS. 5 to 9 show successive stages of a method for manufacturing a number of electronic incense assemblies 100 as shown in FIG. 1, according to an exemplary embodiment. The method includes the following steps:

Step S1: as shown in FIG. 5, providing a mold 50 having a number of molding cavities 501. Each molding cavity 501 is a blind hole and includes a hemispherical molding portion 5011 at the bottom and a cylindrical receiving portion 5012 coaxial with the molding portion 5011 and in communication with the outside of the mold 50. In alternative embodiments, the molding porting 5011 is semi-oval-shaped or cone-shaped.

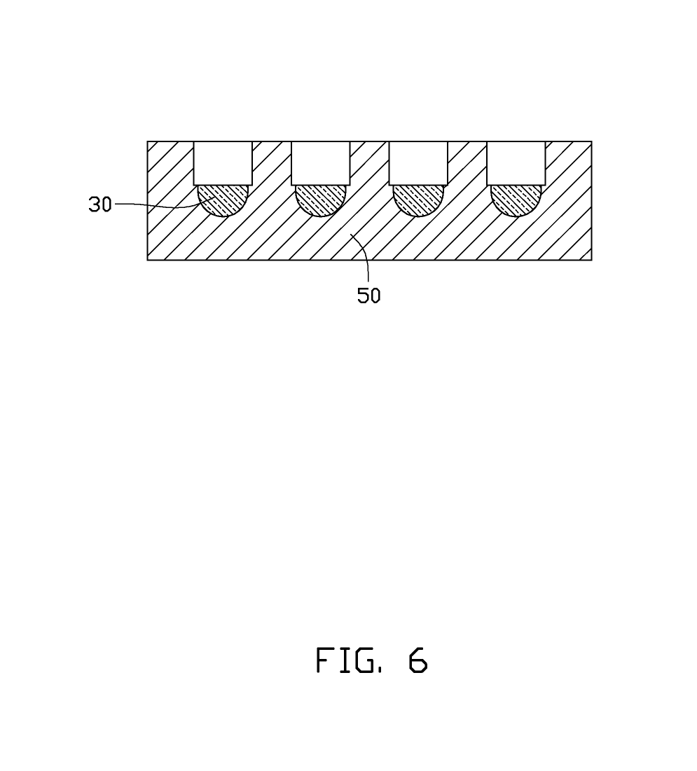

Step S2: as shown in FIG. 6, injecting transparent adhesive into each molding cavity 501. The adhesive is ultraviolet curable adhesive and is fully filled in the molding portion 5011 of each molding cavity 501 to form an incense head member 30 in each molding cavity 501.

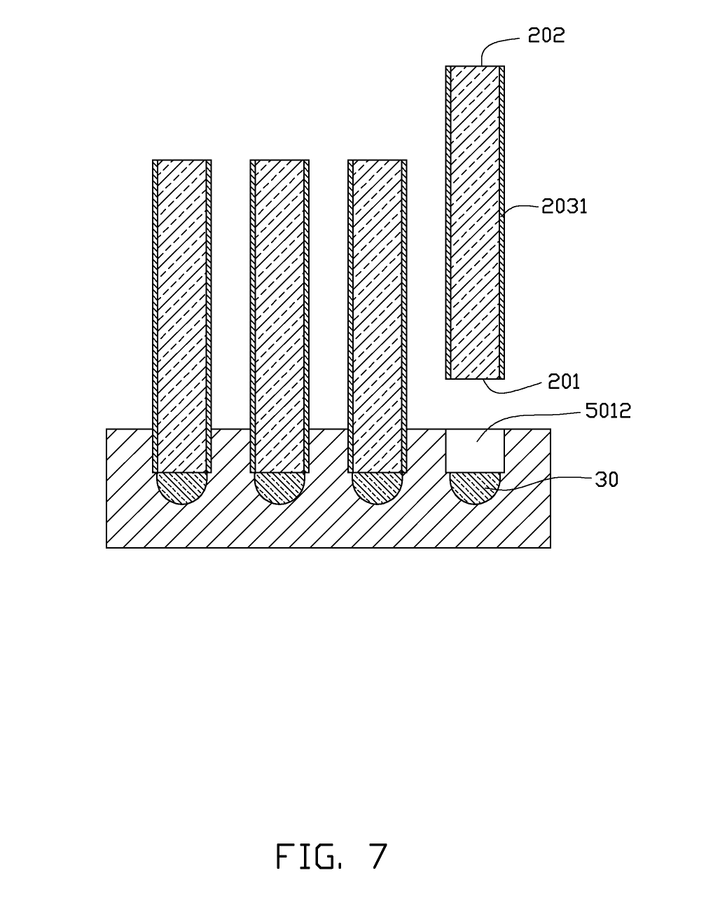

Step S3: as shown in FIG. 7, providing a number of incense sticks 20. The number of the incense sticks 20 corresponds to the number of the molding cavities 501. Each incense stick 20 is made of transparent material and is cylindrical. Each incense stick 20 includes a circular top surface 201, a circular bottom surface 202 opposite to the top surface 201, and a side surface 203 perpendicularly connecting between the top surface 201 and the bottom surface 202. An opaque layer 2031 is coated on the side surface 203. A diameter of each incense stick 20 corresponds to a diameter of the receiving portion 5012. The top surface 201 of one end of each incense stick 20 is inserted into a respective one of the molding cavities 501. Therefore each incense stick 20 is partially received in the receiving portion 5012 of a corresponding molding cavity 501 with the top surface 201 being adhered to the incense head portion 30.

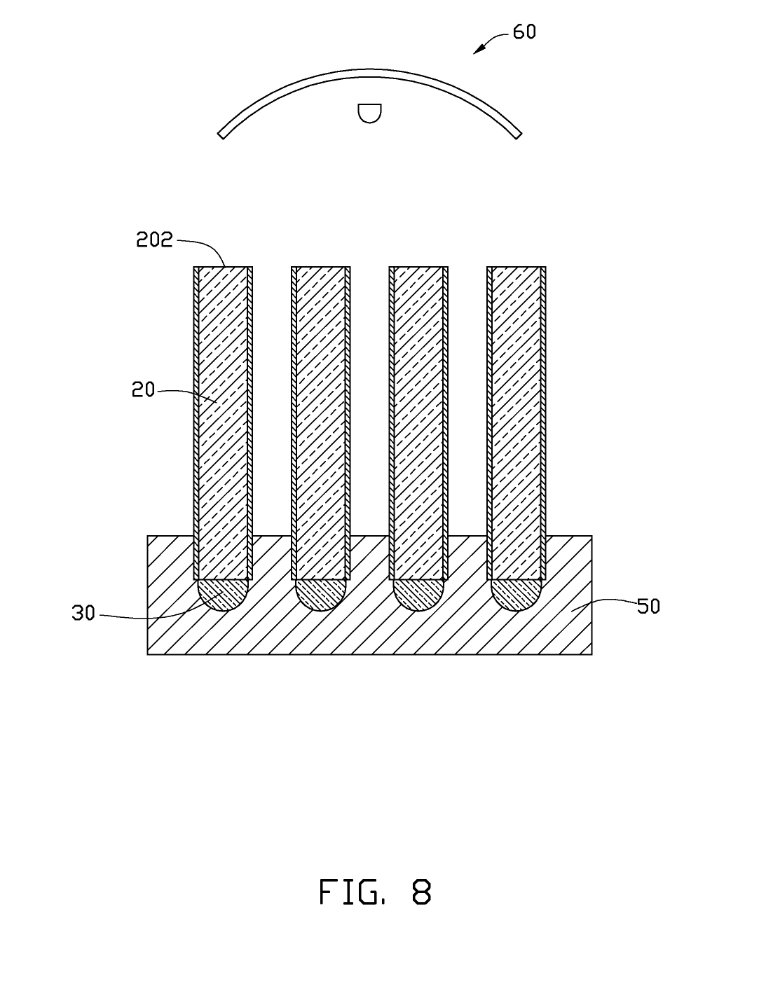

Step S4: as shown in FIG. 8, providing an ultraviolet lamp 60. The ultraviolet lamp 60 is positioned to be spaced from and faces the bottom surface 202 of each incense stick 20. The ultraviolet lamp 60 emits ultraviolet light and projects the ultraviolet light to the bottom surface 202 of each incense stick 20. The ultraviolet light is transmitted to illuminate and cure each incense head member 30 through a corresponding incense stick 20. Each incense head member 30 is cured and molded by the mold 50. Each incense head member 30 is connected to a corresponding incense stick 20.

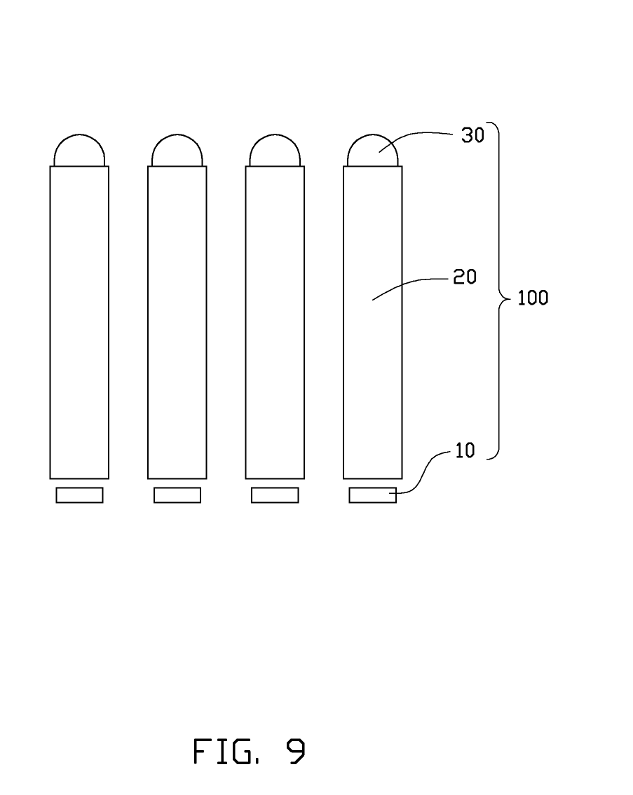

Step S5: as shown in FIG. 9, providing a number of light sources 10. Each incense stick 20 connected to an incense head member 30 is taken out of a corresponding molding cavity 501. Each light source 10 is positioned to be spaced from and face the bottom surface 202. Therefore a number of electronic incense assemblies 100 as shown in FIG. 1 are manufactured.

In alternative embodiments, the number of molding cavities 501 can be at least one. Accordingly, in step S3 at least one incense stick 20 is provided and in step S5 at least one light source 10 is provided.

In alternative embodiments, is step S2, the adhesive can be other adhesive rather than ultraviolet curable adhesive, for example, the adhesive is naturally cured rather than being cured by ultraviolet light. Accordingly, step S4 can be omitted when the adhesive is naturally cured.

In alternative embodiments, the at least one incense stick 20 can be replaced by the incense stick 40 of FIG. 4. The incense stick is a truncated cone in shape, and an area of the top surface 401 of each incense stick is greater than an area of the bottom surface 402 of each incense stick 40. Accordingly, the diameter of the receiving portion 5012 corresponds to a diameter of the top surface 401 of each incense stick 40.

Particular embodiments are shown here and described by way of illustration only. The principles and the features of the present disclosure may be employed in various and numerous embodiments thereof without departing from the scope of the disclosure as claimed. The above-described embodiments illustrate the scope of the disclosure but do not restrict the scope of the disclosure.

What is claimed is:

1. An electronic incense assembly, comprising:
an incense stick comprising a top surface, a bottom surface opposite to the top surface, and a side surface connecting between the top surface and the bottom surface, the side surface being coated with an opaque layer;
a light source facing the bottom surface for emitting light to the bottom surface; and
a molded incense head member adhered on the top surface.

2. The electronic incense assembly as claimed in claim 1, wherein the incense head member is made of transparent adhesive.

3. The electronic incense assembly as claimed in claim 2, wherein the incense head member is made of ultraviolet curable adhesive.

4. The electronic incense assembly as claimed in claim 1, wherein the incense stick is cylindrical and made of a transparent material.

5. The electronic incense assembly as claimed in claim 4, wherein the incense stick is made of a material selected from the group consisting of poly methyl meth acrylate, poly carbonate, silicone, and glass.

6. The electronic incense assembly as claimed in claim 1, wherein the incense stick is a truncated cone in shape, and an area of the top surface is greater than an area of the bottom surface.

7. The electronic incense assembly as claimed in claim 1, wherein the incense head member is hemispherical-shaped, semi-oval-shaped, or cone-shaped.

8. A method for manufacturing at least one electronic incense assembly, the method comprising:
providing a mold having at least one molding cavity, each molding cavity comprising a molding portion at its bottom and a receiving portion coaxial with the molding portion and in communication with the outside of the mold;
injecting adhesive into each molding cavity to full fill the molding portion of each molding cavity and form an incense head member in each molding cavity;
providing at least one incense stick, each incense stick comprising a top surface, a bottom surface opposite to the top surface, and a side surface connecting between the top surface and the bottom surface, the side surface coated with an opaque layer;
inserting each incense stick into a corresponding one of the at least one molding cavity with the top surface being adhered to the incense head member;
providing at least one light source;
taking each incense stick with the incense head member out of the corresponding molding cavity; and
positioning each light source to face the bottom surface of a corresponding incense stick.

9. The method as claim in claim 8, wherein the adhesive is ultraviolet curable adhesive.

10. The method as claim in claim 9, comprising:
positioning an ultraviolet lamp to face the bottom surface of each incense stick; and
activating the ultraviolet lamp to emit ultraviolet light to the bottom surface of each incense stick.

11. The method as claimed in claim 8, wherein the at least one incense stick is cylindrical and made of a transparent material.

12. The method as claimed in claim 8, wherein the at least one incense stick is made of a material selected from the group consisting of poly methyl meth acrylate, poly carbonate, silicone, and glass.

13. The method as claimed in claim 8, wherein the molding portion is hemispherical-shaped, semi-oval-shaped or cone-shaped, and the receiving portion is cylindrical-shaped.

14. The method as claimed in claim 8, wherein the at least one incense stick is a truncated cone in shape, and an area of the top surface of each incense stick is greater than an area of the bottom surface of each incense stick.

15. The method as claimed in claim 14, wherein a diameter of the receiving portion corresponds to a diameter of the top surface.
